(19) United States
(12) Patent Application Publication
(10) Pub. No.: US 2025/0174332 A1
(43) Pub. Date: May 29, 2025

(54) AUTOMATIC PROGRAM GENERATOR FOR SPINAL CORD STIMULATION

(71) Applicant: Boston Scientific Neuromodulation Corporation, Valencia, CA (US)

(72) Inventors: Ismael Huertas Fernandez, Seville (ES); Que T. Doan, West Hills, CA (US)

(73) Assignee: Boston Scientific Neuromodulation Corporation, Valencia, CA (US)

(21) Appl. No.: 18/956,818

(22) Filed: Nov. 22, 2024

Related U.S. Application Data
(60) Provisional application No. 63/603,490, filed on Nov. 28, 2023.

(51) Int. Cl. G16H 20/40; A61N 1/02; A61N 1/05; A61N 1/36; A61N 1/372; G16H 40/60; G16H 50/30

(52) U.S. Cl. CPC G16H 20/40; A61N 1/025; A61N 1/36062; A61N 1/36185; A61N 1/37241; G16H 40/60; G16H 50/30; A61N 1/0551

(57) ABSTRACT

Methods and systems for generating, testing, and evaluating therapy programs for use in neuromodulation. Test programs are generated using paresthesia and/or anatomical considerations. As test programs are applied to the patient, the test programs are evaluated using patient feedback and grading of locations in relation to active therapy delivery electrodes of the system.

### CROSS REFERENCE TO RELATED APPLICATIONS

This application claims the benefit of U.S. Provisional Patent Application Ser. No. 63/603,490, filed on Nov. 28, 2023, and titled AUTOMATIC PROGRAM GENERATOR FOR SPINAL CORD STIMULATION, which is related to copending U.S. Provisional Patent Application Ser. No. 63/603,485, filed on Nov. 28, 2023, and titled ADVANCED ASSISTANT AND USER INTERFACE FOR SPINAL CORD STIMULATION SELF-PROGRAMMING, the disclosures of which are incorporated herein by reference.

### TECHNICAL FIELD

The disclosure is directed to programming and controlling spinal cord stimulation systems. More particularly, the disclosure is directed to methods and systems for patient reprogramming of spinal cord stimulation systems.

### BACKGROUND

Spinal cord stimulation (SCS) is performed by issuing electrical pulses from a lead positioned in the spinal column of a patient, typically to address symptoms related to pain. An implantable pulse generator (IPG) issues the electrical pulses to the lead, and is typically implanted elsewhere in the patient, such as in the buttocks. The electrical pulses are delivered according to a therapy program, which defines which electrodes issue pulses of specified amplitudes and pulse widths at determined repetition rates, as well as other parameters.

The process of developing programs for the electrical pulses can be time-consuming, often including repeated testing of electrical outputs to determine relationships between delivered pulses, electrodes through which the pulses are delivered, and neural response of the patient. Some approaches define a central point of stimulation (CPS), which can be the mathematical “centroid” of output current or voltage, for example (other definitions can be used for CPS). For example, paresthesia mapping may be performed by moving a CPS longitudinally and/or laterally relative to the lead or leads, to determine where paresthesia sensations are perceived by the patient in relation to the location of the CPS. With such mapping completed, the physician then programs the SCS system to deliver therapy where it is needed to address the patient's underlying needs.

It is common for a physician to determine one to four programs, and often only one or two programs, for the patient to use. The patient may, for example, use a patient remote control (RC) to select from available programs stored in the RC and/or IPG, which may be given specific names (such as by naming the program for the target it is designed to treat, for example, left leg, right hand, back, etc.) when stored so that the patient can recognize what is being commanded by the RC. However, the hardware itself may accommodate much more than this; some systems have storage for as many as 16 programs. Moreover, the in-clinic design of a program may be limited by the available time for the process and the knowledge or habits of those supervising it.

Additional and alternative ways of generating programs for SCS are desired.

### OVERVIEW

The present inventors have recognized, among other things, that a problem to be solved is the need for new and/or alternative methods, devices and systems for determining or constructing test programs for use in an implantable medical device system, illustratively used for neural therapy such as spinal cord stimulation.

A first illustrative and non-limiting example takes the form of a method of treating a patient, the patient having a lead implanted therein relative to the spinal cord, the lead carrying a plurality of electrodes, the method comprising: receiving a set of parameters including a reference program for spinal cord stimulation defining a current fractionalization determining utilization of the plurality of electrodes, data related to lead position in the spinal cord of the patient, and an indication of paresthesia location experienced by the patient when the reference program is in use; determining a reference central point of stimulation (CPS) of the reference program; automatically constructing a plurality of test programs, the plurality of test programs including: at least one paresthesia-based test programs each having a test CPS at a distance from the reference CPS; and a set of anatomical-based test programs each using a different subgroup of the plurality of electrodes; wherein each test program defines an active electrode set having at least one electrode to which a current fraction and therapy polarity is assigned and testing at least one of the plurality of test programs by: receiving an input from a patient for testing a selected one of the plurality of test programs; initiating therapy to the patient using the selected one of the plurality of test programs; and receiving a patient score in response to the selected one of the plurality of test programs.

Additionally or alternatively, the method may also include defining a grid of spinal cord coordinates around the lead; and assigning a grid metric to each location on the grid derived from the patient scores. Additionally or alternatively, each grid metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode. Additionally or alternatively, the score is a product of the grade and a patient outcome for the test program. Additionally or alternatively, the grade is determined at each grid location using all active electrodes in each one of the plurality of test programs.

Additionally or alternatively, the at least one at least one paresthesia-based test program uses a CPS that is approximately one to ten millimeters from the reference CPS in one of a lateral direction, a rostral direction, a caudal direction, or a combination of lateral, rostral and caudal directions. Additionally or alternatively, the set of anatomical-based test programs includes one to about four anatomical-based test programs.

Additionally or alternatively, the electrodes include two columns of electrodes, and the anatomical-based test programs allocate current to at least four electrodes in each of the two columns in a side-by-side fashion.

Another illustrative and non-limiting example takes the form of a method of treating a patient, the patient having a lead implanted therein relative to the spinal cord, the lead carrying a plurality of electrodes and attached to an implantable pulse generator (IPG) that communicates with a patient remote control (RC), the IPG storing at least one therapy program therein, the therapy program defining a utilization of the plurality of electrodes, the method comprising: activating a first therapy program and issuing therapy pulses to the patient from the IPG; querying the patient using the RC regarding effects of the first therapy program; receiving a patient response including a first patient score in response to the query; determining, from the patient response, dissatisfaction with the first therapy program; determining a reference central point of stimulation (CPS) of the first therapy program; automatically constructing a plurality of test programs, the plurality of test programs including a first test program having a test CPS at a predetermined distance from the reference CPS; testing at least one of the plurality of test programs by: receiving an input from a patient for testing the first test program; and initiating therapy to the patient using the first test program; querying the patient using the RC regarding effects of the first test program; receiving a second patient score from the patient in response to delivery of therapy using the first test program.

Additionally or alternatively, the method also includes determining that the second patient score exceeds the first patient score, and replacing the first therapy program with the first test program for use in treating the patient.

Additionally or alternatively, testing at least one of the plurality of test programs includes testing at least a second test program; and the method further includes receiving a third patient score from the patient in response to delivery of therapy using the second test program, determining which of the first, second and third patient scores is highest, and: if the first patient score is highest, either automatically constructing more test programs, or testing at least a third test program; or either determining that the second patient score is highest, and replacing the first therapy program with the first test program for use in treating the patient; or determining that the third patient score is highest, and replacing the first therapy program with the second test program for use in treating the patient.

Additionally or alternatively, the step of automatically constructing a plurality of test programs, is performed by a clinician programmer (CP), wherein the method includes: the CP issuing data defining the first therapy program by communicating with the IPG; the CP performing the step of determining the reference CPS; the CP, after determining the reference CPS and automatically constructing the plurality of test programs, issuing data defining the plurality of test programs to the IPG; and the IPG storing the plurality of test programs.

Additionally or alternatively, the step of automatically constructing a plurality of test programs, is performed by the RC, wherein the method includes: the RC performing the step of determining the reference CPS; the RC, after automatically constructing the plurality of test programs, issuing data defining the first test program to the IPG; and the IPG storing the first test programs.

Additionally or alternatively, the RC performs the automatically constructing and issuing data steps in response to the RC determining, from the patient response, dissatisfaction with the first therapy program.

Another illustrative and non-limiting example takes the form of a method of treating a patient, the patient having a lead implanted therein relative to the spinal cord, the lead carrying a plurality of electrodes and attached to an implantable pulse generator (IPG) that communicates with a patient remote control (RC), the IPG storing at least one therapy program therein, the therapy program defining a utilization of the plurality of electrodes, the method comprising: activating a first therapy program and issuing therapy pulses to the patient from the IPG; querying the patient using the RC regarding effects of the first therapy program; receiving a patient response including a first patient score in response to the query; determining, from the patient response, dissatisfaction with the first therapy program; automatically constructing a plurality of test programs, the plurality of test programs including a first test program and a second test program, each being an anatomical-based test program using a different subgroup of the plurality of electrodes; testing at least two of the plurality of test programs by: receiving an input from a patient for testing the first test program; initiating therapy to the patient using the first test program; querying the patient using the RC regarding effects of the first test program; receiving a first patient test score from the patient in response to delivery of therapy using the first test program; receiving an input from a patient for testing the second test program; initiating therapy to the patient using the second test program; querying the patient using the RC regarding effects of the second test program; and receiving a second patient test score from the patient in response to delivery of therapy using the second test program.

Additionally or alternatively, the method includes defining a grid of spinal cord coordinates around the lead; and assigning a grid metric to each location on the grid derived from the patient scores.

Additionally or alternatively, each test program defines a set of active electrodes each receiving a fraction of a total current having a polarity, and each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode.

Additionally or alternatively, the grid metric for each location on the grid is a sum of products of the grade and a patient test score outcome for each test program that has been tested.

Additionally or alternatively, the grade is determined at each grid location using all active electrodes in each test program that has been tested.

Additionally or alternatively, the electrodes include two columns of electrodes, and each test program test program allocate currents to at least four electrodes in each of the two columns in a side-by-side fashion.

Additionally or alternatively, the step of automatically constructing a plurality of test programs is performed by a remote resource in communication with at least one of a bedside monitor or a patient remote controller, and the plurality of test programs are communicated by the remote resource to the at least one of the bedside monitor or the patient controller for loading to the implantable pulse generator.

Another illustrative and non-limiting example takes the form of a patient remote control for an implantable medical device system, the medical device system including an implantable pulse generator (IPG) and a lead, the pulse generator containing pulse generating circuitry for outputting neural therapy, the patient remote control comprising: a user interface for interacting with a user or patient; a communications circuitry for communicating with the IPG; a controller and associated memory, the memory storing controller readable instructions in non-transitory form for the following: receiving a reference program for neural stimulation, the reference program defining a current fractionalization determining utilization of the plurality of electrodes, data related to lead position in the patient, and an indication of paresthesia location experienced by the patient when the reference program is in use, determining a reference central point of stimulation (CPS) of the reference program; generating a plurality of test programs, wherein each test program defines an active electrode set having at least one electrode to which a current fraction and therapy polarity is assigned, at least one of the test programs having a different CPS than the reference CPS; instructing the pulse generator to test at least one of the plurality of test programs by: receiving, at the user interface, an input from a patient for testing a selected one of the plurality of test programs; using the communications circuitry to instruct the IPG to initiate therapy to the patient using the selected one of the plurality of test programs; and receiving, at the user interface, a patient score in response to the selected one of the plurality of test programs; and determining, based on the received patient scores, whether to replace the reference program with a selected one of the test programs or to generate additional test programs.

Additionally or alternatively, the at least one paresthesia-based test program each uses a test CPS at a distance from the reference CPS.

Additionally or alternatively, the at least one at least one paresthesia-based test program uses a CPS that is approximately one to ten millimeters from the reference CPS in one of a lateral direction, a rostral direction, a caudal direction, or a combination of lateral, rostral and caudal directions.

Additionally or alternatively, the test programs include at least one anatomical-based test programs each using a different subgroup of the plurality of electrodes.

Additionally or alternatively, the controller readable instructions include instructions for: defining a grid of spinal cord coordinates around the lead; assigning a grid metric to each location on the grid derived from the patient scores; and using the grid metrics to generate additional test programs after at least one of the plurality of test programs is tested.

Additionally or alternatively, each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode.

Additionally or alternatively, each metric is a product of the grade and a patient score for the test program.

Another illustrative and non-limiting example takes the form of a test program generation system for use with an implantable medical device system, the medical device system including a patient remote control, an implantable pulse generator (IPG) and a lead, the pulse generator containing pulse generating circuitry for outputting neural therapy, the patient remote control including a user interface for interacting with a user or patient and communications circuitry for communicating with the IPG; the test program generation system configured to: receive a reference program for neural stimulation, the reference program defining a current fractionalization determining utilization of the plurality of electrodes, data related to lead position in the patient, and an indication of paresthesia location experienced by the patient when the reference program is in use, identify a reference central point of stimulation (CPS) of the reference program; generate a plurality of test programs, wherein each test program defines an active electrode set having at least one electrode to which a current fraction and therapy polarity is assigned, at least one of the test programs having a different CPS than the reference CPS; use the remote control to instruct the pulse generator to test at least one of the plurality of test programs by: receiving, at the user interface, an input from a patient for testing a selected one of the plurality of test programs; using the communications circuitry to instruct the IPG to initiate therapy to the patient using the selected one of the plurality of test programs; and receiving, at the user interface, a patient score in response to the selected one of the plurality of test programs; and determine, based on the received patient scores, whether to replace the reference program with a selected one of the test programs or to generate additional test programs.

Additionally or alternatively, the at least one test program includes a plurality of paresthesia-based test programs each having a test CPS at a distance from the reference CPS. Additionally or alternatively, the at least one test program includes a set of one to at least four anatomical-based test programs each using a different subgroup of the plurality of electrodes.

Additionally or alternatively, the test program generation system may also be configured to define a grid of spinal cord coordinates around the lead; assign a grid metric to each location on the grid derived from the patient scores; and use the grid metrics to generate additional test programs after at least one of the plurality of test programs is tested.

Additionally or alternatively, each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode, and the grid metric is a product of a grade for the grid point and a patient score for the test program.

Additionally or alternatively, further comprising a clinician programmer which performs each of the receive, identify, generate, and determine steps, the clinician programmer being in communication with the patient remote control or a bedside monitor that periodically communicates with the IPG.

Additionally or alternatively, the test generation system may include a remote resource which performs each of the receive, identify, generate, and determine steps, the clinician programmer being in communication with the patient remote control or a bedside monitor that periodically communicates with the IPG.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows components of an illustrative neuromodulation system;

FIG. 2 shows an illustrative pulse generator and lead system;

FIG. 3 shows a spinal cord stimulation system as implanted in a patient;

FIG. 4 shows an illustrative grid approach to analyzing SCS;

FIG. 5 illustrates several anatomical-based test therapies;

FIGS. 6A-6C show several paresthesia-based test therapies;

FIG. 7 illustrates program storage and data in a neuromodulation system;

FIG. 8 is a chart of patient data;

FIG. 9 illustrates patient scoring of a reference and anatomy-based test therapies;

FIG. 10 illustrates grading of grid locations for a neuromodulation therapy;

FIG. 11 illustrates scoring of grid locations using grading and patient input;

FIG. 12 is an illustrative block flow diagram for a method; and

FIG. 13 illustrates communication among system components.

### DETAILED DESCRIPTION

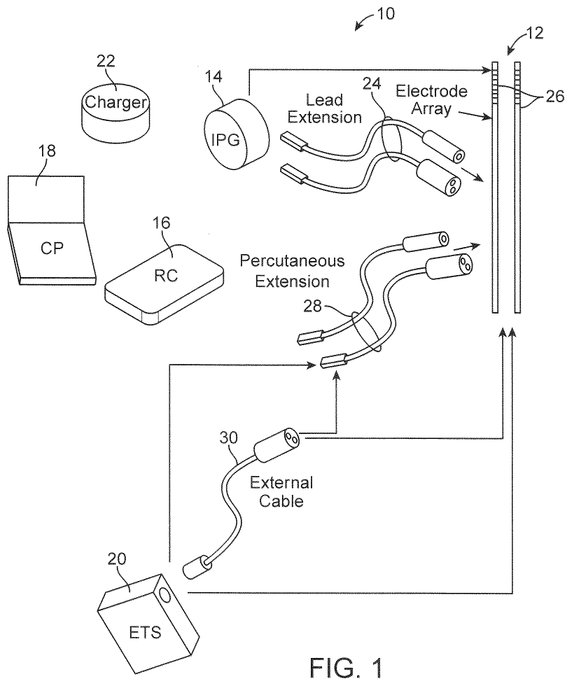

FIG. 1 shows a system for providing neurological therapy, which may be used, for example, as a system for spinal cord stimulation (SCS), deep brain stimulation (DBS), peripheral nerve stimulation (PNS), or functional electrical stimulation (FES). Several examples herein are directed to SCS, but other uses may be had. The system 10 includes leads 12 configured for coupling to an implantable pulse generator (IPG) 14. The IPG may communicate with one or more of a patient remote control (RC) 16, a clinician programmer (CP) 18, and/or a charger 22. An external testing system (ETS) 20 may also be provided for testing therapy parameters prior to implantation of the IPG, using percutaneous extensions 28 and, as needed, an external cable 30 to couple to the leads 12. The use of an ETS may be referred to as a trial period. If needed, lead extensions 24 may be used to couple the IPG to the leads 12.

As shown in FIG. 1, the leads 12 may include arrays of electrode contacts on linear leads 26. In other examples, paddle leads may be used. One, two or even four leads 12 may be provided, with up to 32 contacts on the leads 12, plus an additional contact in the form of the housing of IPG 14. More or fewer contacts on more or fewer leads may be provided depending the particular system.

The IPG 14 can couple directly to the leads 12 or may be coupled via the lead extensions 24, depending on the positioning of each element as implanted. The IPG 14 may include a rechargeable battery and charging coil to allow recharging when placed in proximity to the charger 22. Alternatively, the IPG may use a non-rechargeable battery and omit a charging coil and charger 22 from the system. In some examples, the IPG 14 may be externally powered and omits a battery entirely.

The CP 18 can be used by a physician to program and/or manipulate the outputs of the IPG 14 and/or ETS 20. For example, the CP 18 can be used by the physician to define a therapy regimen or program for application to the patient. A program may, for example, define the individual electrical pulses (pulse width, shape and/or amplitude as desired), a repetition rate or frequency for the pulses, and any additional parameters such as, for example and without limitation, definition of bursts of pulses, ramping at the start of therapy, duration of therapy or quantity of pulses, and any patient adjustable parameters. A burst, for example, may include a set of closely coupled (in time) pulses separated by a relatively longer quiescent period between bursts. Patient adjustable parameters may allow a patient to, for example, adjust therapy amplitude up or down. As illustrated, there may be any number of leads and numerous electrodes. A therapy program may also define which electrodes are active/inactive during pulse delivery, and may specify different pulse amplitudes from one electrode to the next. Therapy can be current controlled or voltage controlled, for example and without limitation.

The CP 18 may be a custom device and/or may take the form of a laptop or tablet computer, for example and without limitation. Multiple programs may be facilitated and stored by the IPG 14 or ETS 20; in some examples, the RC 16 may instead or in addition store the programs to be used. Communication amongst the IPG 14, RC 16, CP 18, ETS 20 and Charger 22 may use any suitable protocol such as wireless RF telemetry, Medradio, inductive communication, Bluetooth, etc.

The RC 16 may be used by a patient to enable or disable therapy programs, to select between available programs, and/or to modify the programs that are available for use. For example, in some embodiments a patient may use the RC 16 to activate a stored program and then manipulate therapy by increasing or decreasing therapy strength and/or changing therapy location, within limits set by the physician. The RC 16 may be a custom device, or may be, for example, a smartphone or tablet having an application thereon for use with the medical device system 10.

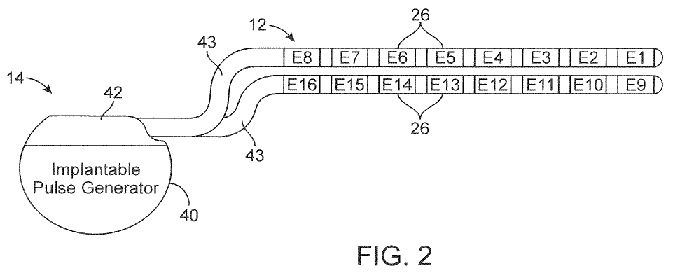

FIG. 2 shows an implantable stimulator and leads. As shown in the closer detail here, the IPG 14 may include a canister 40 and header 42. The canister 40 is conductive in most examples, using biocompatible materials such as titanium and/or stainless steel, for example, to allow use as an electrode when implanted. The header 42 allows removable connection to the lead 12, which in this example may have a bifurcation or yoke allowing two segments 43 to extend therefrom, to two arrays 26 at the distal end of the lead 12. A common structure for securing the leads 12 is the use of a setscrew in the header 42. The electrode arrays 26 can be numbered as shown to facilitate case of understanding when programming, with, for example, one array marked electrodes E1 to E8 on one of the leads 43, with E1 being distalmost. Other conventions may be used.

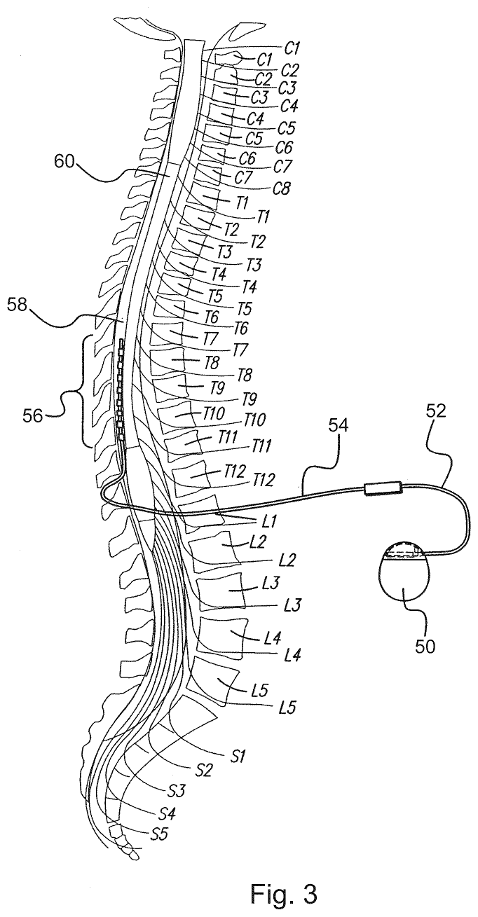

FIG. 3 shows an illustrative spinal cord stimulation system as implanted. In this example, an IPG 50 may be placed near the buttocks or in the abdomen of the patient, with or without a lead extension 52 for coupling to the lead(s) 54 that enter the spinal column. Region 56, at about the level of the lower thoracic or upper lumbar vertebrae may serve as an entry point to the spinal column, where the distal end of the lead 54 with an electrode array may be placed close to the spinal cord 58. Other locations for the IPG 50 and/or lead 54 may be used.

The standard approach to therapy in systems similar to those shown in FIGS. 1-3 has been that the IPG 14, 50, or ETS 20, may offer current controlled or voltage-controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

Multiple current or voltage sources may be used for therapy outputs. For example, fields that result, and the shape of the volume of tissue that will respond to neuromodulation can be controlled by delivering different voltage or current amounts to the available electrodes. Multiple independent current control (MICC) may be used, for example. MICC is a stimulus control system that provides a plurality of independently generated output currents that may each have an independent quantity of current. The use of MICC can allow spatially selective fields to be created by therapy outputs. The term “fractionalization” may refer to how the total current issued by the pulse generator via the electrodes is divided up amongst the electrodes of the lead and/or including the pulse generator canister, which can serve as an additional electrode.

Some examples of current or prior versions of IPG circuitry, including in particular the stimulation circuitry but also power, input/output, and microcontroller, as well as planned future examples, may be found in U.S. Pat. No. 10,716,932, the disclosure of which is incorporated herein by reference. The system may include, for example and without limitation, a controller in the form of a microcontroller or microprocessor, with associated, auxiliary or peripheral circuitry including logic, amplifiers, filters, etc. for processing received signals, as well as a communication circuitry for communicating using any suitable wireless or other mode. A memory may be included and linked to the controller to store in a non-transient form controller or otherwise machine readable instructions encoding the methods described below.

Pulse generator circuitry may include that of the various commercially known implantable pulse generators for spinal cord stimulation, Vagus nerve stimulation, and deep brain stimulation as are also well known. Additional examples of controllers, circuitry, designs and operation of system devices (IPG, CP, RC, Charger, and ETS, for example) can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The capabilities of many SCS systems extend far beyond how such systems are commonly used. For example, a patient may undergo therapy programming following implantation to test a small number of programs to determine which programs are best suited to the patient. Each such program is manually configured by the physician (which may include configuration at the direction of a physician), often to align the regions in which a patient experiences paresthesia with body areas in which the patient experiences pain. Supra-threshold therapies may be configured so that the patient experiences paresthesia overlapping pain during therapy. Sub-threshold therapies may be configured so that paresthesia is not experienced during therapy, but may be used to set up the therapy initially. For example, a subthreshold therapy can be configured to issuing therapy to cause paresthesia to overlap pain regions, and then reducing therapy intensity (as by reducing amplitude, or pulse width, for example) until the paresthesia ceases to be perceived while pain remains suppressed. The patient may go home at the end of the implantation and associated follow up clinical visits with one or two programs configured in the IPG and selectable and/or accessible with the RC, where each program is manually configured. New and alternative approaches to stimulation programming are desired.

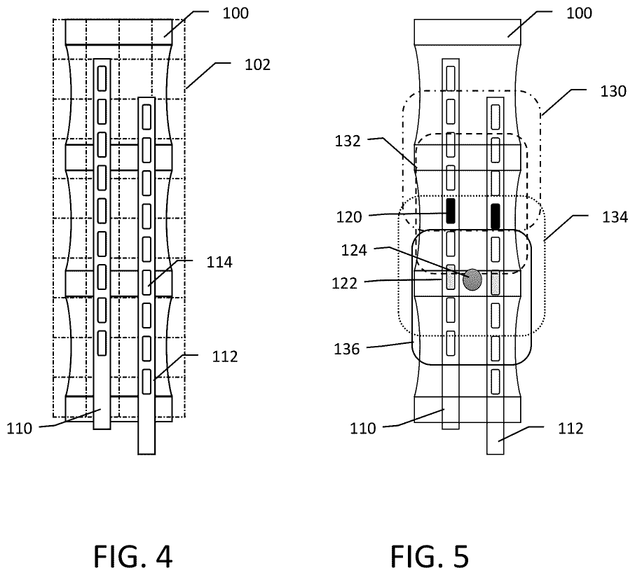

FIG. 4 shows an illustrative grid approach to analyzing SCS. A vertebral structure is shown at 100, with leads 110 and 112 shown relative to the structure 100. Each lead has a plurality of electrodes 114 thereon. A grid 102 represents a standard space of spinal cord coordinates, and is used to guide the system in defining and evaluating alternative or “rescue” programs to be used by the patient if the patient's original therapy programs are not providing effective results. The grid 102 thus comprises a plurality of cells, shown as a 4-by-10 grid in the Figure but not limited to such dimensions. Other grid definitions can be used recognizing the tradeoff between desired higher resolution and less desired greater computational power and memory of having more or fewer cells.

Imaging and other data, such as X-rays and any other imaging taken during an implant procedure and/or after implantation of leads 110, 112, may be used to place the leads 110 and 112 relative to the grid 102, and to tailor how the vertebral structure 100 is positioned relative to each of the grid 102 and the leads 110, 112. Some implantable neuromodulation systems have the capability to determine or estimate the relative positions of leads, as well as to monitor for any lead migration post-implant, as described for example, in U.S. Pat. Nos. 6,993,384, 7,831,307, 7,853,330, 8,131,357, 8,401,665, 8,233,992, 8,380,301, and 8,700,165, the disclosures of which are incorporated herein by reference. For example, using methods described in such patents, inter-electrode impedance or electric field potential measurements can be used to determine the relative orientation of one lead to other leads in the spinal column or other body/tissue location.

A “sweet spot” for therapy may exist for a given patient. The sweet spot is the stimulation location and electrical field configuration that provides the patient with effective, or most effective, therapy outcomes. One or multiple sweet spots may exist for a given patient. Over time, the sweet spot may change with respect to the lead position as well as location relative to the anatomy. Changes may occur due to lead movement, or due to changes in the neural tissue response to electrical currents and fields, or for various other reasons. In some patients, temporary inflammation may cause the nerve and/or neural target to move, for example left or right, relative to midline. Even if such movement is limited to a few millimeters, the sweet spot may move enough to justify reprogramming.

Regardless why a change takes place, the intent is to respond to such changes by providing the patient additional, automated, and possibly self-administered, enhancements. Each cell in the grid can be used in various steps below to apply grades to therapy outputs indicating the grid-cell's contribution to therapy, and combine such grades with patient outcome indications.

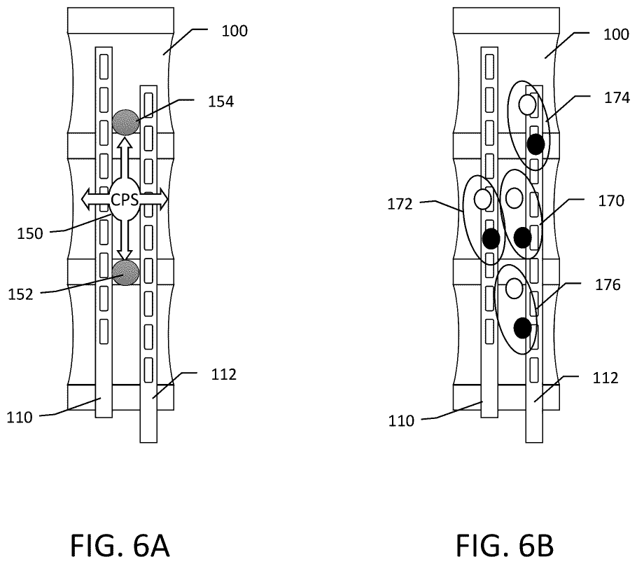

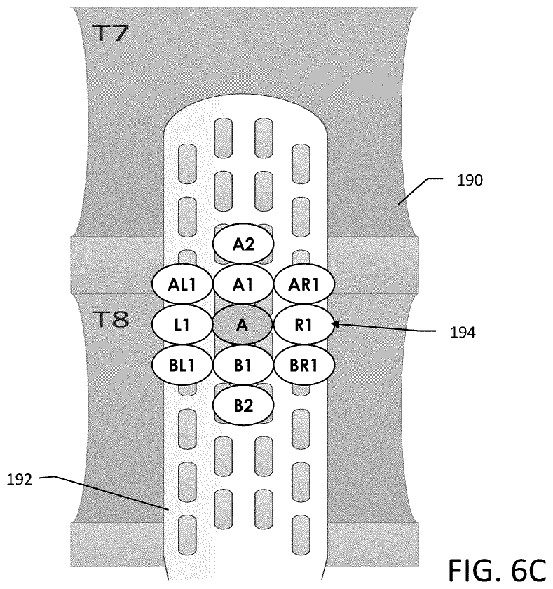

Test therapy programs can be defined as shown in FIGS. 5 and 6A-6C. The system (IPG and RC for example, operating cooperatively but not necessarily requiring clinician intervention) may define these test therapy programs. In other examples, the CP may automatically define the test therapy programs. The system may also use remote resources, such as a central server operated by a manufacturer or clinical practice, as desired to automatically, semi-automatically, or manually define the test therapy programs. The remote resource may be fully automated, or may be directed, controlled, or supervised by an individual, such as a company representative, a nurse, a physician, etc. Further, some “semi-automatic” systems may determine proposed changes and then (remotely) contact an individual (a company representative, a nurse, a physician, etc.) to approve potential test programs, to approve reprogramming or other changes to previously stored programs, and/or to provide other inputs as needed.

FIG. 5 shows how several anatomically-based test programs can be generated. In the illustrative example, the patient has a reference program—that is, one configured by or with guidance or approval of a physician during a clinical follow up. The physician may use a CP to specifically identify a reference program. In other examples, a reference program can be determined by an RC or IPG over time, based on patient usage and/or selection among available programs; that is, a physician may provide the patient with a number of possible programs to use, and the patient may use the RC to choose and activate programs. A program which is most used by the patient may become the reference program over time. In another example, a program which receives a highest rating from the patient—that is, a program indicated as “best” by patient selection or feedback—may be identified as a reference program. Alternatively, as noted, the physician may make such a selection of a reference or master program via the user interface of a CP at a clinical follow-up.

The reference program in this illustration issues electrical signals using anodes shown in black at 120, and cathodes shown in grey at 122, with a central point of stimulation (CPS) at 124. In the illustration, the CPS is cathodic and so is located between the cathodes 122; there may instead be only an anodic CPS, both anodic and cathodic CPS, or an overall CPS, if desired.

In some examples, the RC, CP, IPG, or a remote resource, infrastructure or server (whether automated, semi-automated, or under control of an individual) may then define a plurality of anatomical test programs. Such definition of test programs proceeds automatically, and does not need physician interaction in some examples. The test programs are illustrated at boxes 130, 132, 134, and 136, each indicating electrode selection for an anatomical-based test program and progressing from caudal to rostral along the structure 100. The anatomical-based test programs can be generated without regard for the position of anode or cathodes in the reference program in some examples. In other examples, the anatomical-based test programs have a relationship to the reference program, such as by having a first test program defined about the CPS, for example, the cathodic CPS 124, of the reference program. Then the system defines additional test programs including one or more that are rostral or caudal thereto with separation of, about 1 to about 10 mm, for example, or any of 3, 6 or 9 mm, or some other distance. For example, in FIG. 5, block 134 is generally centered at the reference program CPS, with the remaining blocks 130, 132, 136 offset by a predetermined distance.

In some examples, the RC and/or IPG share functions for performing the test program definitions while the patient is not at a clinic. In some examples, a CP may automatically calculate a set of test programs during a clinical follow-up and load the test programs to the IPG and/or RC for later use as needed (such as in response to patient dissatisfaction with programs in use). In some examples, a remote infrastructure may define test programs, such as on a remote server. Candidate programs can be generated remotely, and a stack of candidate programs may be loaded to the RC or IPG such as by communication with a bedside monitor, which receives programs from the remote serve such as via Internet, cellular, WiFi, or other connection, and loads test program data to an RC or IPG during monitoring communication sessions. If the RC receives the test programs, it may communicate such programs to the IPG as needed, such as in response to a patient request to try one of the test programs, or may communicate the test programs during communications with the IPG for any other purpose. In other examples, remotely generated programs can be communicated to an RC by cellular or other means, including via Internet connection, WiFi, etc.

Electrode utilization in each box 130, 132, 134, 136 may be similar to provide an initial configuration set. In a non-limiting illustration, using two linear leads as shown, there may be eight electrodes in each of the boxes 130, 132, 134, 136, and these may be identified as right and left, and from 1 to 4, as shown here:

| | |
|---|---|
| Left_1 | Right_1 |
| Left_2 | Right_2 |
| Left_3 | Right_3 |
| Left_4 | Right_4 |

The boxes can be larger or smaller, and may operate only on one of the leads, if desired. The basic idea is to obtain data across more of the anatomy at least initially, and to tailor down the data gathering over time if deemed appropriate. If the leads 110 and 112 are not vertically aligned, the boxes may be defined to use anatomically level (or close to level) electrodes. For example, as shown, treating the tip electrode as E1 for lead 110, and the tip electrode as E9 for lead 112 (as in FIG. 2), box 130 would use electrodes as follows:

| | | | |
|---|---|---|---|
| Left_1 | E2 | Right_1 | E9 |
| Left_2 | E3 | Right_2 | E10 |
| Left_3 | E4 | Right_3 | E10 |
| Left_4 | E5 | Right_4 | E11 |

Test therapy for box 130 can be allocated (fractionalized) across the electrodes in any suitable way. A pre-determined configuration, assuming no patient information for example, may be as shown here:

| | | | |
|---|---|---|---|
| Left_1 | +25 | Right_1 | +25 |
| Left_2 | +25 | Right_2 | +25 |
| Left_3 | −25 | Right_3 | −25 |
| Left_4 | −25 | Right_4 | −25 |

Where each number is a percentage of the total current delivered. Another approach may spread field somewhat more:

| | | | |
|---|---|---|---|
| Left_1 | +33 | Right_1 | +33 |
| Left_2 | +17 | Right_2 | +17 |
| Left_3 | −17 | Right_3 | −17 |
| Left_4 | −33 | Right_4 | −33 |

Other current fractionalizations may be used, if desired. If voltage controlled outputs are used, the voltages may be flat and fixed, or may vary from one electrode to another. For example,

| | | | |
|---|---|---|---|
| Left_1 | +V | Right_1 | +V |
| Left_2 | +V | Right_2 | +V |
| Left_3 | −V | Right_3 | −V |
| Left_4 | −V | Right_4 | −V |

Where V is a reference amplitude. Other voltage distributions can be used as desired.

Other examples may expand the above to a paddle electrode. If a paddle electrode with two columns is used, the preceding examples can be used. A four-column paddle may be configured as shown above by moving the program squares to the left, center or right side of the paddle, as can be readily understood. Alternatively, three or four column test programs can be used analogous to the preceding.

FIGS. 6A-6C show additional examples of test programs. Again, leads 110 and 112 are shown relative to the vertebral structure 100 in each Figure. Here, rather than an anatomical approach, a reference-based or paresthesia-based set of test or rescue programs is defined. The CPS 150 of the reference program is used as the defining point for the automatic generation of the test programs. As before, the CPS 150 may be a cathodic CPS, anodic CPS, or overall CPS. In each instance, the CPS 150 may be the centroid of the relevant subset of current or voltage, or of the overall current, voltage, or power. An “overall” CPS, for example, may be determined as the mathematical centroid of output power or output current/voltage magnitudes. A cathodic CPS may consider only cathodic current or voltage, and anodic CPS may consider only anodic current or voltage.

In the illustration of FIG. 6A, for example, the CPS 150 may be adjusted down as shown at 152, or up, as shown a 154. Right or left movement may be achieved as well, such as shown in FIG. 6B, with an original reference program having anode and cathode CPS as illustrated at 170, with adjustments to any of positions 172, 174, 176. Movement to the right or left is limited by the most lateral electrodes, and up or down by the top or bottom electrodes. Each new CPS is based on the prior CPS being moved in a direction and for a distance in these examples. Each new CPS may be located, for example, about 1 to about 10 millimeters in any direction from the original CPS. For example, 7 millimeters may be a distance in the up/down directions, and 2 millimeters in the lateral directions, though other distances and directions (for example, diagonals such as both left and up, or right and down, etc.) can be used. The rescue program CPS may set or calibrated by keeping or approximating the original fractionalization distributing current around the new CPS. Alternatively, the current or voltage output may instead be spread or narrowed, such as by changing fractionalization. In some examples, an anodic CPS and cathodic CPS is defined, and only one of the two is changed, if desired. Tailoring, with spread or narrowing of current fractionalization, or adjustment of only one of the CPS, may be part of fine tuning after an initial pass of developing and testing paresthesia-based therapies has been completed, if desired.

FIG. 6C shows another illustrative example. Here, the spinal anatomy is shown at 190, including thoracic vertebrae T7 to T8. A paddle lead 192 overlies the spinal anatomy, and is illustrated, in this example, as having four columns of eight electrodes each, for a total of 32 electrodes. An original programmed CPS is shown as A, in the darkened oval in the center of a set of potential CPS locations illustrated at 194. Using A as the starting program, a set of test programs surrounding the original CPS, A, are illustrated. These additional programs are located at spacings round the original CPS, A, and programs for the additional programs may be stored in the system, for example, as follows:

| Program Slot | Program Name | Description |
|---|---|---|
| 1 | User1 | User Defined Program 1 |
| 2 | User2 | User Defined Program 2 |
| 3 | User3 | User Defined Program 3 |
| 4 | User4 | User Defined Program 4 |
| 5 | User5 | User Defined Program 5 |
| 6 | User6 | User Defined Program 6 |
| 7 | A2 | A2: 4 mm above |
| 8 | B2 | B2: 4 mm below |
| 9 | AL1 | AL1: 2 mm above, 1.5 mm left |
| 10 | A1 | A1: 2 mm above |
| 11 | AR1 | AR1: 2 mm above, 1.5 mm right |
| 12 | L1 | L1: 1.5 mm left |
| 13 | R1 | R1: 1.5 mm right |
| 14 | BL1 | BL1: 2 mm below, 1.5 mm left |
| 15 | B1 | B1: 2 mm below |
| 16 | BR1 | BL2: 4 mm below, 1.5 mm right |

Ten additional CPS-driven programs are thus defined. Program slots 7-16, for many patients and users, would be unused, and so storing the alternative programs 7-16 in program slots 7-16 does not interfere with the ordinary programming of the system. In the example, the preset algorithm generates the set of additional locations where available. Using the paddle lead example, if, for example, the original program was at an edge of the rightmost or leftmost column, fewer than ten alternative programs may be generated, as the CPS cannot move left of the leftmost column of electrode contacts, for example. It may also be noted that movement in the rostral/caudal direction in this example uses a larger spacing than in the lateral direction. That is, for example, when moving the CPS left or right, the step size int his example is 1.5 mm, while rostral caudal step size is larger at 2 mm. Such a differentiation is optional, but can be useful in context, as neural stimulation along the spine is often more sensitive to lateral (left/right) changes than to vertical (rostral/caudal) changes.

In some examples, prior paresthesia testing data may be stored by the device or system and used to determine starting points for developing new programs. For example, if paresthesia mapping was performed by starting at the right border of the available electrodes (rightmost column of a paddle, for example) and stopped before reaching the left border, new therapy locations may target those areas that have not previously been tested. This approach can be generalized to include the closest locations separate from those previously tested, so that a greater area of the available locations of CPS can be tested.

If programs are set in additional slots, some examples may identify the presence of additional user programs and either seek permission from the user (physician for example) before replacing a program, or may not create and store all the alternative programs at once. In operation, the patient would be presented the opportunity to initiate a testing sequence, and the patient's feedback regarding which of the additional programs provides effective relief of symptoms would be used to set the new programmed parameters.

FIG. 7 illustrates program storage and data in a neuromodulation system. In FIG. 7, the IPG program memory is divided into three sections. A first section 200 contains slots of memory for up to seven physician programmed programs, P1 to P7. A second section 202 contains slots of memory for up to five anatomical rescue or test programs, P8-P12. A third section 204 contains slots of memory for up to four paresthesia-based rescue or test programs. Other allocations may be used, as desired.

Each program, as indicated at 206, may define pulses, P, steering, S, aggregate instructions A, and configuration instructions, C, which may be used as described in U.S. Pat. No. 10,716,932, the disclosure of which is incorporated herein by reference. For example, the configuration instruction C may control overall, indicating which aggregate instructions are to be used. Each aggregate instruction may combine pulse definitions and steering settings (including fractionalization) and a quantity of repeats to be performed as part of the aggregate instructions. Other program structures can be used, as desired.

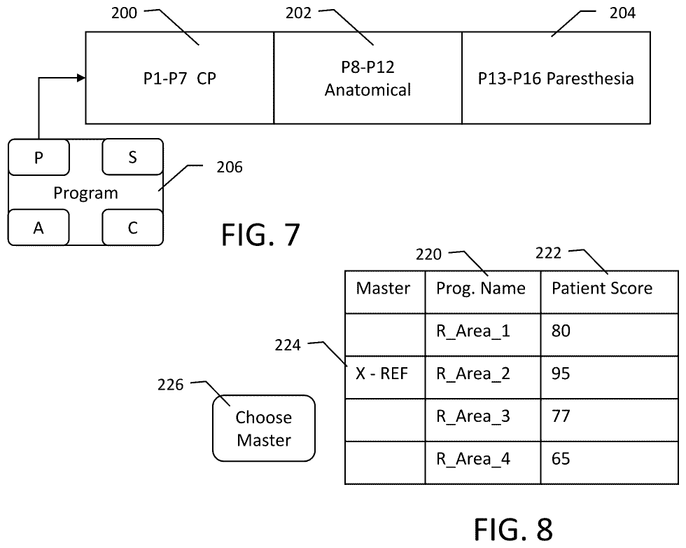

FIG. 8 is a chart of patient data. A set of programs, which may be clinician-defined programs, are named as indicated at 220. As the patient uses each program, the RC may obtain patient feedback, such as a patient score as indicated at 222 telling the RC which program is best performing in the patient's opinion. Patient scores 222 may instead or in addition use objective criteria, such as blood pressure and/or heart rate while the program is running (where lower heart rate or blood pressure may indicate effective pain relief), or performance measures (accelerometer-sensed patient movement, where more movement indicates better pain relief, for example). Other inputs can be used for the patient scores 222, as desired, and may be scaled or normalized as desired to allow comparison of very different measures in a single analysis.

The patient “score” obtained by patient feedback may indicate a range of patient experience. A low score (for example, less than 2 on a 0-5 scale) may indicate the patient is experiencing a side effect of therapy during a particular therapy program execution, such as undesired levels of paresthesia, burning or painful sensations, nausea, muscle stimulation, or other undesired effects. A middle-level score (2 to 3 on a 0-5 scale) may indicate the absence of side effects but also limited to no benefits. A higher score (4 to 5 on a 0-5 scale) may indicate therapy benefits (reduced pain sensations for example) with minimal to no side effects. Other scales and approaches may be used, such as by having multiple patient “scores” entered for example to separate side effect reporting from clinical benefit reporting.

A program having a highest patient score 222 can be identified as the reference program, as indicated at 224. Reference program 224 may instead be chosen by a physician, such as by selecting an icon as shown at 226 presented on the user interface of a CP. Reference program 224 may be chosen instead by a patient directly by providing an icon as shown at 226 and a list as shown at 220/222/224 on an RC user interface. A patient may indirectly identify a master program by making other choices, for example, if the patient has several programs available for use, the program which the patient selects most often, or uses for the greatest duration of time, may be identified by the IPG or RC as a reference program.

The reference program may then be used in the preceding examples to define the anatomically-based and paresthesia-based (CPS-based) test programs or rescue programs. The test programs can be generated, as indicated previously, by a CP during a device interrogation such as may occur during a follow up visit. The test programs may instead be generated by the RC operating in conjunction with the IPG. Test programs may be generated remotely instead, if desired. In each instance, the patient score 222 can be used, if desired, to determine the reference program 224. If the patient is only given a single program to use when the IPG is programmed, the single program becomes the reference program.

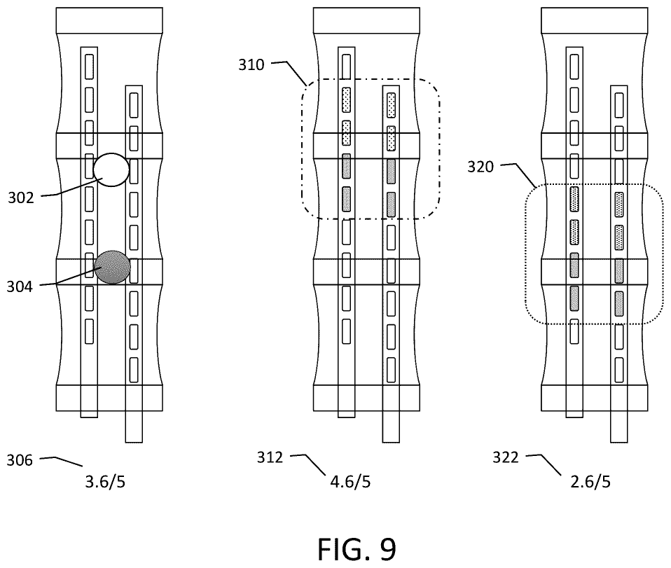

FIG. 9 illustrates patient scoring of a reference and two anatomical-based test therapies. A reference program configuration is illustrated to the left side of FIG. 9, with an anodic CPS at 302 and the cathodic CPS in grey at 306. The patient has given this therapy a score of 3.6/5, that is, a relatively lower score than may be desired for a given therapy output.

In response, the system has tested two alternative therapies, using anatomically based test therapies at 310 and 320. The cathodes of each of the test therapies 310, 320 are shown in grey, with dot-fill for the anodes, thus repeating the general configuration of the original therapy. The anode/cathode configuration can be reversed in some examples, defining a further set of test programs. Here, one of the test programs, at 310, scores higher (4.6/5, as shown at 312) than the original program 302/304, as well as being higher than the other test program 320, as indicated at 322. The program at 310 may replace the original program, in some examples as a default program, and also as a reference program. In other examples, the improved score for test program 310 may trigger further fine tuning, such as by moving the CPS (one, the other, or both of 302, 304) in the direction of program 310—that is, up, as shown in FIG. 9.

While some examples use a locally managed stack of test programs, such as using an RC and/or IPG to determine recommendations, a system may instead use a remotely managed stack of test programs. For example, communication with the IPG through a bedside monitor or RC may be used to report back usage of test programs and patient responses thereto. Combinations of performance measures and/or similarity to a reference program may be used to manage the stack to prioritize test programs yielding positive feedback or which are similar (such as in terms of CPS location, or stimulated region) to a reference program may be used as well. When considering patient feedback, aging may be used to de-prioritize patient feedback that is older relative to newer or more recent patient feedback.

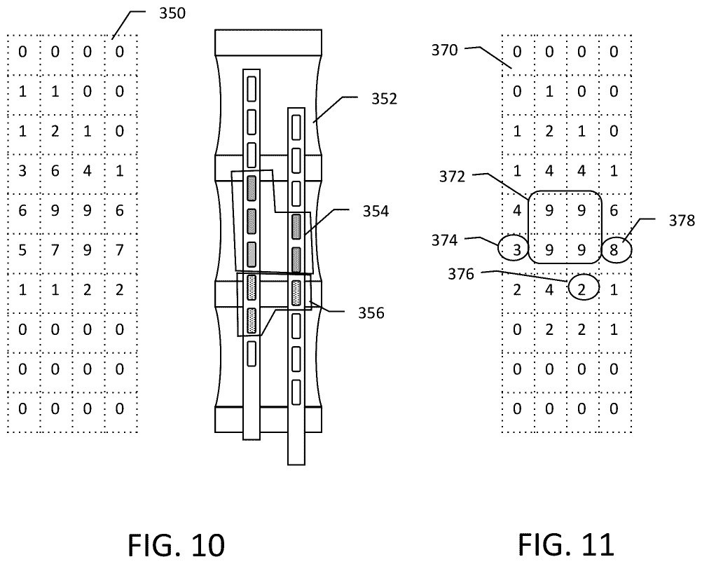

FIG. 10 illustrates grading of grid locations for a neuromodulation therapy. Grid locations are shown in FIG. 10 at 350, corresponding to the cathodes of a therapy tested as shown at 352. Other examples may use active electrodes as a whole group, or anodes only, if desired, in place of cathodes. The grades shown in the grid 350 may be determined using the cathodes indicated in grey at 354 of the tested therapy. Thus, for example, at each cell in the grid 350, a coordinate-related grade (CRG) is determined as a function of several inputs. This formula is one example:

CRG (x,y)=f (polarity, pulse type, current fraction, distance to contact).

As a further example:

CRG_{c}(x,y)=w_{c}*wₜ*Σdᵢ*iᵢ

Where the subscript, c, indicates cathode, weight we is a weight applied to cathodic grades, wₜ is a weight based on the type of therapy pulse (active recharge or passive recharge, for example), and the summation adds up the current of each i^{th} electrode used as a cathode times distance from the x,y cell of each cathodic electrode. The summation may be limited to only those electrodes within a predefined distance of the grid location, or only the one, two, three, or four closest active electrodes, if desired, or may apply to all electrodes. An analogous grade can be given for anodes 356, if desired. An active recharge therapy pulse includes a first phase having a first amplitude and pulse width, and a second phase having an equal amplitude and pulse width as the first phase, but opposite polarity, as contrasted with a passive recharge therapy pulse that lacks a second phase at all and instead relies on shorting the electrodes to ground in order to remove post-stimulation polarization. An active recharge therapy pulse type, in some examples, may be applied a larger weight, such as double, triple or more, as compared a passive recharge therapy pulse.

The grade may instead be a summation:

$${CRG}_{c} ( x , y ) = {w}_{c} + {w}_{t} + \sum {d}_{i} * {i}_{i}$$

Here, the first two weights can be as before, as is the summation, though the weights here will be selected to normalize the use of the current/distance summation, as it would be desired that the summation provide a relatively stronger influence on the grade.

As used herein, a grade is the analysis of electrical and spatial characteristics of the output therapy, relative to the grid and grid points. A score on the other hand is derived from a patient response, including objective (heart rate, blood pressure, etc.) and subjective (patient assessment of therapy and/or side effects) measures. The grid metric represents a combination of both. A grid metric may be understood as representing both the “utilization” of a particular grid point as indicated by distance, output type, and amplitude/power of a given output, and the patient response to such utilization.

The CRG can then be used to determine a grid metric for each grid point, using the patient score, as shown here:

Metric(x,y)=Pt_Score*CRG(x,y)

Again, separate metrics can be determined for each grid location as both anode and cathode, if desired. Alternatively, the grades and metrics can be power-based, or may use current magnitudes, if desired, to provide a single metric. In some examples, only cathodic data are used to determine an initial cathodic CPS, and fine tuning may involve adjusting the anodic CPS. In other examples, a guarded cathode approach is taken, in which a cathodic CPS is surrounded by anodes which may be generally equally distributed. In still other examples, a monopolar therapy can be issued with the IPG canister serving as anode. In each such example, the use of the terms anode and cathode may refer to the polarity of the first phase of the output therapy, if active recharge is used, or the only phase of output therapy, if passive recharge is used. Grid coordinates can be converted into a heat map if desired, based on location performance.

FIG. 11 illustrates metrics of grid locations using grading and patient input. While integer metrics are shown, it may be understood that this is merely for simplicity of illustration, and more significant digits can be used in a given implementation. The metrics, as previously described, are shown. Such metrics can be determined by applying the preceding discussion of grid metrics to test programs and, if desired, the reference program or any other programs that the patient has used. A receding horizon may be used when considering patient scores. That is, for a given grid location, assuming a plurality of patient scores are available for a plurality of programs, an aggregate score can be determined:

$$Agg . Score = {S}_{1} + ( 1 - λ ) * {S}_{2} + {( 1 - λ )}^{2} * {S}_{3} + {( 1 - λ )}^{3} * {S}_{4}$$

Where scores S1, S2 are scores ordered temporally, such that older scores are given lower weight in the summation due to the receding horizon parameter, λ. Other formulations can be used, such by creating an explicit time-based discount factor:

$$Agg . Score = D F ( {t}_{1} ) {S}_{1} + D F ( {t}_{2} ) * {S}_{2} … D F ( {t}_{n} ) * {S}_{n}$$

Where the discount factor, DF, is a function of the time, tₙ, since the nₜₕ score was obtained. Again, other formulations can be used, with the general idea being that older scores have lesser influence on the aggregate score for a given grid location.

The result is that the grid 370 is populated with grid metrics at each grid point. In the example, the numbers may represent cathodic data, based on the combination of stimulus grades, patient scores, and any desired aging factors. The grid 370 may then be used to identify hotspots or sweet-spots for therapy. For example, box 372 indicates locations of increased likelihood of patient benefit for therapy using any of these locations. A therapy program placing a cathodic CPS in box 372 may then be generated automatically by an RC, CP, IPG and/or remote server. Additionally, cold-spots can be identified as well. For example, locations 374 and 376 may be ones to avoid, while location 378 may be one to test as well when fine-tuning the resulting therapy. A heat map can be generated, if desired.

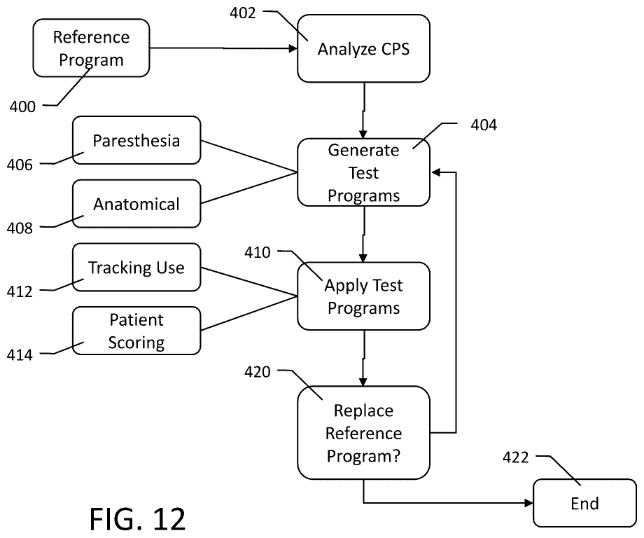

FIG. 12 is an illustrative block flow diagram for a method. At block 400, a reference program for the patient is identified. The CPS of the reference program is determined at 402. That reference CPS is used to generate test programs, as indicated at 404. The test programs can be paresthesia based 406 or anatomically based 408. As described above, test programs that are paresthesia based 406 may be generated by moving the CPS. Test programs that are anatomically based 408 may be generated by moving overall stimulation up and/or down, using the reference program CPS as a starting place but not necessarily defining programs based on CPS movement.

The test programs are then applied 410. Test programs 410 may be applied by the patient selecting test programs for use via the user interface of a remote control. This may be done as set forth in copending U.S. Prov. Pat. App. No. 63/603,485, filed on Nov. 28, 2023, and titled ADVANCED ASSISTANT AND USER INTERFACE FOR SPINAL CORD STIMULATION SELF-PROGRAMMING, the disclosure of which is incorporated herein by reference. The use of test programs can be tracked as indicated at 412, to, for example, suggest that the patient use or try each test program. Patient scoring is obtained as indicated at 414. It may be noted that block 404 can be performed in clinic under supervision of a physician, or other trained personnel, while block 410 may be performed by the patient acting independently, in some examples. In other examples, block 404 can be executed automatically, for example in response the patient's input. Application of test programs 410 may also be performed with physician or other trained personnel supervising, if desired.

In an illustrative example, the test programs are tested for a limited amount of time, in the range of 1-2 minutes, or 1-5 minutes (longer durations may be used if desired). The patient can provide ratings as a feedback loop. As the testing continues, the patient may be asked to provide therapy ratings and/or indicate when “recapture” of the original therapy efficacy is achieved.

Whether to replace the reference program, as indicated at 420, may be determined by the system finding that a test program performs better according to patient scores than the reference program. The process may be iterative and return to block 404 after block 420, if desired. For example, after tracking use 412 and getting patient scores 414, the grid analysis shown previously in FIGS. 10-11 may be used to assess and generate further test programs in block 404. If a replacement program is determined and tested to the satisfaction of the patient, the process may end as indicated at 422. In some examples, “satisfaction of the patient” may be explicit by asking the patient if the replacement program is satisfactory, or may be determined using patient scores.

Step 410 may include testing all or a subset of the generated test programs. For example, in some embodiments, patent scores are monitored as the testing progresses and compared against a first threshold score indicative of a very good therapy result or high patient satisfaction and, if the first threshold score is exceeded by a test program, the testing may stop and the last tested program replaces the reference program automatically. In other examples, at least a threshold number of test programs are applied at 410 before the method proceeds to 420 to determine whether to replace the reference program. Results can be updated continuously or at intervals, such as daily, until either a stop criteria is met, if desired. Though not shown in FIG. 12, there may be a recurring loop back from 420 to 410 as testing progresses, if desired.

If the system reverts from block 420 to block 404, the generation of test programs may be performed via anatomical 408 reference using a grid as calculated in FIGS. 10-11. The metrics at each grid point (FIG. 11) can be used to determine new test programs from this anatomical-based grid. For example, using FIG. 11, the grid points clustered at 372 can be used to identify a virtual CPS, and the system then uses the current fractionalization process to calculate current outputs (anode and cathode) that will place the CPS in the desired grid points shown in 372. The position of the leads and electrodes relative to the grid will then be used to generate a fractionalization for one or more test programs within the highest scoring region of the grid. The surrounding scores may also be used, for example, to avoid delivering current to the low scoring adjacent locations at 374 and 376, while providing current at 378, for example. Best-fit type analysis can be used to automatically generate the fractionalization, along with a model of surrounding tissue. For use in the spinal column, for example, the underlying tissue model may include a modeled conductance/impedance that would be encountered in the surrounding tissue. Additionally, known data, such as gathered from impedance testing, can also be used to model current flows and other behavior in the system.

The method of FIG. 12 may include an additional entry condition. It may be noted that the reference program will often be one that has been subject to previous testing, whether in-clinic or by the method of FIG. 12. In some examples, the patient remote control may not allow the patient to select a test program until the patient has indicated dissatisfaction with the reference program. This may be a way to prevent the patient from putting the into an undesirable configuration in which therapy benefits are lost by misuse, for example. Further, in some examples, the IPG and/or RC may be configured to retain the last physician-approved reference program in memory for subsequent follow-ups in-clinic, so that previously tested and set configurations may not be lost due to patient use of the system testing capabilities.

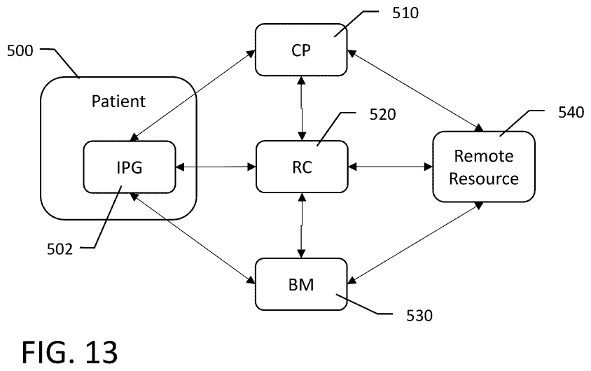

FIG. 13 shows communication among system components, and provides an additional discussion of the above concepts. A patient 500 has an implanted IPG 502. The IPG 502 may be configured to communicate with any of a CP 510, RC 520, and/or bedside monitor (BM) 530. The communications with the IPG may use any suitable mode, including, for example and without limitation, Bluetooth, Bluetooth Low Energy, Medradio, ISM, inductive, optical, and/or sonic communication. For example, the CP 510 may communicate with the IPG 502 during a programming session in the clinical context. The RC 520 may communicate with the IPG 502 whenever the patient uses the RC 520 or brings the RC 520 into proximity with the IPG 502. The BM 530 may be designed to communicate from time to time with the IPG 502, such as when the patient is at rest. The CP 510, RC 520 and BM 530 each may also communicate to a remote resource, which may be a remote server, infrastructure or any other suitably connected system. The communication links to the remote resource 540 may be via Internet, cellular, or other suitable mode. The CP 510 and RC 520, as well as the RC 520 and BM 530, may also communicate with one another, such as by Bluetooth, Medradio, ISM, WiFi, cellular, etc. or even over the internet. These various communication links are illustrative and not limiting; other links and link types may be used.

In some examples, the CP 510 may define test programs. During a programming session in-clinic, a physician using the CP 510 may select a reference program for use in defining test programs. Such test programs can be loaded into the IPG 502 or RC 520 from the CP 510. The CP 510 may instead communicate such programs to the remote resource 540, which can communicate the test programs to the RC 520 and/or BM 530 when desired or requested, or automatically wherein such test programs are stored for later use or download to the IPG 502. When test programs are determined by the CP 510, the physician may, optionally, be asked to provide approval or review of such test programs.

In some examples, the RC 520 or BM 530 may define test programs, using a reference program as identified by a physician using the CP 510 and/or as identified by patient use or selection, and set by the RC 520 or IPG 502, as described above. Test programs can then be communicated to the IPG 502, automatically or when requested or needed, such as after a patient has indicated dissatisfaction with programs in use, or simply for purposes of potential testing or availability even absent any indication of dissatisfaction. If a test program is found to be better for therapy purposes than a previous reference program, the test program may replace the previous reference program as a new reference program. The test programs defined by an RC 520 or BM 530 may also be communicated to the CP 510 or remote resource 540 for purposes of approving the use of these test programs and/or to confirm selection of a new reference program.

In some examples, the IPG 502 may defined test programs, using a reference program as identified by a physician using the CP 510 and/or as identified by patient use or selection, as described above. Such test programs may be communicated to the RC 520 to allow the patient to select which test program to use at a given time, if desired. The RC 520 can then also be used to obtain patient feedback to determine which test programs function for their intended purpose, and/or to select a new reference program. Test programs, and, if desired, a new reference program may be communicated to the RC 520 and/or BM 530 for further communication to the CP 510 and/or remote resource 540, for example, where the test programs or new reference program can be reviewed and approved.

In some examples, the remote resource 540 may defined test programs, using a reference program as identified by a physician using the CP 510 and/or as identified by patient use or selection, as described above. Here, the remote resource 540 may be fully automated in its decisions, or may be semi-automated and uses human input (such as from a nurse, company representative, or physician) in some or all circumstances. The remote resource 540 may have available to it additional data, such as population-based data to identify other patients similarly situated to patient 500. “Similarly situated” may include patients with similar symptoms, age, illness, clinical/health background, etc. “Similarly situated” may also include patients having reference programs that are similar to the reference program of patient 500, if desired.

Test program selection and implementation in some examples may occur when requested by the RC 520 using patient inputs to determine whether and when testing is to occur. By driving the test program selection and implementation with the RC 520, using patient direction, the patient's input can be obtained as well via the RC 520, providing a closed loop for feedback. Test programs may be generated, on the other hand, whenever desired by any of the system components, as described above.

An illustrative example may take the form of a patient remote control for an implantable medical device system, the medical device system including an implantable pulse generator (IPG) and a lead, the pulse generator containing pulse generating circuitry for outputting neural therapy, the patient remote control comprising: a user interface for interacting with a user or patient; a communications circuitry for communicating with the IPG; a controller and associated memory comprising: first means (402) configured for receiving and analyzing a reference program (400) for neural stimulation defining a current fractionalization determining utilization of the plurality of electrodes, data related to lead position in the patient, and an indication of paresthesia location experienced by the patient when the reference program is in use, the first means (402) further configured to determine a reference central point of stimulation (CPS) of the reference program. Such a first means may be realized as a software or firmware module, application, instruction set, and/or object which can perform as described above relative to block 402 of FIG. 12. The patient remote control may further include second means (404) configured to automatically generate a plurality of test programs, wherein each test program defines an active electrode set having at least one electrode to which a current fraction and therapy polarity is assigned, at least one of the test programs having a different CPS than the reference CPS. Such a second means may be realized as a software or firmware module, application, instruction set, and/or object which can perform as described above relative to block 404 of FIG. 12. The patient remote control may also include third means (410) configured for instructing the IPG to test at least one of the plurality of test programs by: receiving, at the user interface, an input from a patient for testing a selected one of the plurality of test programs; using the communications circuitry to instruct the IPG to initiate therapy to the patient using the selected one of the plurality of test programs; and receiving, at the user interface, a patient score in response to the selected one of the plurality of test programs. Such a third means may be realized as a software or firmware module, application, instruction set, and/or object which can perform as described above relative to block 410 of FIG. 12. The patient remote control may also include fourth means (420) configured for determining, based on one or more received patient scores from the third means, whether to replace the reference program with a selected one of the test programs or to request the first means generate additional test programs. Such a fourth means may be realized as a software or firmware module, application, instruction set, and/or object which can perform as described above relative to block 420 of FIG. 12.

A test program generation system for use with an implantable medical device system, the medical device system including a patient remote control, an implantable pulse generator (IPG) and a lead, the pulse generator containing pulse generating circuitry for outputting neural therapy, the patient remote control including a user interface for interacting with a user or patient and communications circuitry for communicating with the IPG; the test program generation system comprising: first means (402) configured for receiving and analyzing a reference program (400) for neural stimulation defining a current fractionalization determining utilization of the plurality of electrodes, data related to lead position in the patient, and an indication of paresthesia location experienced by the patient when the reference program is in use, the first means (402) further configured to determine a reference central point of stimulation (CPS) of the reference program. Such a first means may be realized as a software or firmware module, application, instruction set, and/or object which can perform as described above relative to block 402 of FIG. 12. Further, the test program generation system may include second means configured to automatically generate a plurality of test programs, wherein each test program defines an active electrode set having at least one electrode to which a current fraction and therapy polarity is assigned, at least one of the test programs having a different CPS than the reference CPS. Such a second means may be realized as a software or firmware module, application, instruction set, and/or object which can perform as described above relative to block 404 of FIG. 12. The test program generation system may include third means, the third means being part of the patient remote control, configured for instructing the pulse generating circuitry to test at least one of the plurality of test programs by: receiving, at the user interface, an input from a patient for testing a selected one of the plurality of test programs; using the communications circuitry to instruct the IPG to initiate therapy to the patient using the selected one of the plurality of test programs; and receiving, at the user interface, a patient score in response to the selected one of the plurality of test programs; Such a third means may be realized as a software or firmware module, application, instruction set, and/or object which can perform as described above relative to block 410 of FIG. 12. The test program generation system may also include fourth means (420) configured for determining, based on one or more received patient scores from the third means, whether to replace the reference program with a selected one of the test programs or to request the first means generate additional test programs. Such a fourth means may be realized as a software or firmware module, application, instruction set, and/or object which can perform as described above relative to block 420 of FIG. 12.

The test program generation system may be configured in several ways as described relative to FIG. 13. For example, the first means, second means, and fourth means are each part of a remote resource (540) or clinician programmer (510) in communication with the patient remote control or a bedside monitor that periodically communicates with the IPG. In some examples, the patient remote control, or the test program generation system, wherein the IPG comprises a memory configured to store a plurality of therapeutic programs for use in delivering therapy to the patient, at least one of the therapeutic program is the reference program, and at least one of the therapeutic program is a test program, wherein the patient remote control does not allow the patient to select a test program until the patient has indicated dissatisfaction with the reference program.

Additionally or alternatively for a patient remote control or test program generation system, the second means is configured to generate at least one paresthesia-based test programs each having a test CPS at a distance from the reference CPS. Additionally or alternatively for a patient remote control or test program generation system, the second means is configured such that the at least one at least one paresthesia-based test program uses a CPS that is approximately one to ten millimeters from the reference CPS in one of a lateral direction, a rostral direction, a caudal direction, or a combination of lateral, rostral and caudal directions. For example, the second means may be further configured to operate using methods as in FIGS. 6A-6C.

Additionally or alternatively for a patient remote control or test program generation system, the second means is configured to generate a set of one to at least four anatomical-based test programs each using a different subgroup of the plurality of electrodes. For example, the second means may be configured to operate using methods as in FIG. 5.

Additionally or alternatively for a patient remote control or test program generation system, the fourth means is configured to define a grid of spinal cord coordinates around the lead, and assign a grid metric to each location on the grid derived from the patient scores; and the second means is configured to use the grid metrics to generate additional test programs after at least one of the plurality of test programs is tested. Additionally or alternatively for a patient remote control or test program generation system, the fourth means is configured such that each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode. Additionally or alternatively for a patient remote control or test program generation system, the fourth means is configured such that each metric is a product of the grade and a patient score for the test program. For example, the fourth means may be configured to operate using methods as in FIGS. 10-11 and described in association therewith.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), cither with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient, the patient having a lead implanted therein relative to the spinal cord, the lead carrying a plurality of electrodes, the method comprising:
receiving a set of parameters including a reference program for spinal cord stimulation defining a current fractionalization determining utilization of the plurality of electrodes, data related to lead position in the spinal cord of the patient, and an indication of paresthesia location experienced by the patient when the reference program is in use;
determining a reference central point of stimulation (CPS) of the reference program;
automatically constructing a plurality of test programs, the plurality of test programs including:
at least one paresthesia-based test programs each having a test CPS at a distance from the reference CPS; and
a set of anatomical-based test programs each using a different subgroup of the plurality of electrodes;
wherein each test program defines an active electrode set having at least one electrode to which a current fraction and therapy polarity is assigned and testing at least one of the plurality of test programs by:
receiving an input from a patient for testing a selected one of the plurality of test programs;
initiating therapy to the patient using the selected one of the plurality of test programs; and
receiving a patient score in response to the selected one of the plurality of test programs.

2. The method of claim 1, further comprising:
defining a grid of spinal cord coordinates around the lead; and
assigning a grid metric to each location on the grid derived from the patient scores.

3. The method of claim 2, wherein each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode.

4. The method of claim 3, wherein the score is a product of the grade and a patient outcome for the test program.

5. The method of claim 3, wherein the grade is determined at each grid location using all active electrodes in each one of the plurality of test programs.

6. The method of claim 1, wherein the at least one at least one paresthesia-based test program uses a CPS that is approximately one to ten millimeters from the reference CPS in one of a lateral direction, a rostral direction, a caudal direction, or a combination of lateral, rostral and caudal directions.

7. The method of claim 1, wherein the set of anatomical-based test programs includes one to about four anatomical-based test programs.

8. The method of claim 6, wherein the electrodes include two columns of electrodes, and the anatomical-based test programs allocate current to at least four electrodes in each of the two columns in a side-by-side fashion.

9. A method of treating a patient, the patient having a lead implanted therein relative to the spinal cord, the lead carrying a plurality of electrodes and attached to an implantable pulse generator (IPG) that communicates with a patient remote control (RC), the IPG storing at least one therapy program therein, the therapy program defining a utilization of the plurality of electrodes, the method comprising:
activating a first therapy program and issuing therapy pulses to the patient from the IPG;
querying the patient using the RC regarding effects of the first therapy program;
receiving a patient response including a first patient score in response to the query;
determining, from the patient response, dissatisfaction with the first therapy program;
determining a reference central point of stimulation (CPS) of the first therapy program;
automatically constructing a plurality of test programs, the plurality of test programs including a first test program having a test CPS at a predetermined distance from the reference CPS;
testing at least one of the plurality of test programs by:
receiving an input from a patient for testing the first test program; and
initiating therapy to the patient using the first test program;
querying the patient using the RC regarding effects of the first test program;
receiving a second patient score from the patient in response to delivery of therapy using the first test program.

10. The method of claim 9, further comprising determining that the second patient score exceeds the first patient score, and replacing the first therapy program with the first test program for use in treating the patient.

11. The method of claim 9, wherein testing at least one of the plurality of test programs includes testing at least a second test program; and the method further includes receiving a third patient score from the patient in response to delivery of therapy using the second test program, determining which of the first, second and third patient scores is highest, and:
if the first patient score is highest, either automatically constructing more test programs, or testing at least a third test program; or either
determining that the second patient score is highest, and replacing the first therapy program with the first test program for use in treating the patient; or
determining that the third patient score is highest, and replacing the first therapy program with the second test program for use in treating the patient.

12. The method of claim 9, wherein the step of automatically constructing a plurality of test programs, is performed by a clinician programmer (CP), wherein the method includes:
the CP issuing data defining the first therapy program by communicating with the IPG;
the CP performing the step of determining the reference CPS;
the CP, after determining the reference CPS and automatically constructing the plurality of test programs, issuing data defining the plurality of test programs to the IPG; and
the IPG storing the plurality of test programs.

13. The method of claim 9, wherein the step of automatically constructing a plurality of test programs, is performed by the RC, wherein the method includes:
the RC performing the step of determining the reference CPS;
the RC, after automatically constructing the plurality of test programs, issuing data defining the first test program to the IPG; and
the IPG storing the first test programs.

14. The method of claim 13, wherein the RC performs the automatically constructing and issuing data steps in response to the RC determining, from the patient response, dissatisfaction with the first therapy program.

15. A method of treating a patient, the patient having a lead implanted therein relative to the spinal cord, the lead carrying a plurality of electrodes and attached to an implantable pulse generator (IPG) that communicates with a patient remote control (RC), the IPG storing at least one therapy program therein, the therapy program defining a utilization of the plurality of electrodes, the method comprising:
activating a first therapy program and issuing therapy pulses to the patient from the IPG;
querying the patient using the RC regarding effects of the first therapy program;
receiving a patient response including a first patient score in response to the query;
determining, from the patient response, dissatisfaction with the first therapy program;
automatically constructing a plurality of test programs, the plurality of test programs including a first test program and a second test program, each being an anatomical-based test program using a different subgroup of the plurality of electrodes; and
testing at least two of the plurality of test programs by:
receiving an input from a patient for testing the first test program;
initiating therapy to the patient using the first test program;
querying the patient using the RC regarding effects of the first test program;
receiving a first patient test score from the patient in response to delivery of therapy using the first test program;
receiving an input from a patient for testing the second test program;
initiating therapy to the patient using the second test program;
querying the patient using the RC regarding effects of the second test program; and
receiving a second patient test score from the patient in response to delivery of therapy using the second test program.

16. The method of claim 15, further comprising:
defining a grid of spinal cord coordinates around the lead; and
assigning a grid metric to each location on the grid derived from the patient scores.

17. The method of claim 15, wherein each test program defines a set of active electrodes each receiving a fraction of a total current having a polarity, and each metric includes a grade for each one of the plurality of test programs which has been tested, the grade determined using, for at least one active electrode in each one of the plurality of test programs which has been tested, a distance to the at least one active electrode, a therapy polarity of the at least one active electrode, and a current fraction of the at least one active electrode.

18. The method of claim 17, wherein the grid metric for each location on the grid is a sum of products of the grade and a patient test score outcome for each test program that has been tested.

19. The method of claim 17, wherein the grade is determined at each grid location using all active electrodes in each test program that has been tested.

20. The method of claim 15, wherein the electrodes include two columns of electrodes, and each test program test program allocate currents to at least four electrodes in each of the two columns in a side-by-side fashion.
